(19) United States
(12) Patent Application Publication
(10) Pub. No.: US 2026/0224883 A1
(43) Pub. Date: Aug. 6, 2026

(54) METHODS AND APPARATUS FOR ESTIMATING A TORQUE ANGLE OF A SYNCHRONOUS ELECTRIC MOTOR

(71) Applicant: Abiomed, Inc., Danvers, MA (US)

(72) Inventor: Hisham Hafez, Danvers, MA (US)

(73) Assignee: Abiomed, Inc., Danvers, MA (US)

(21) Appl. No.: 19/465,233

(22) Filed: Jan. 30, 2026

Related U.S. Application Data
(60) Provisional application No. 63/752,047, filed on Jan. 31, 2025.

(51) Int. Cl. A61M 60/538; A61M 60/122; A61M 60/411

(52) U.S. Cl. CPC A61M 60/538; A61M 60/122; A61M 60/411; A61M 2205/103; A61M 2205/3327; A61M 2205/502

(57) ABSTRACT

Methods and apparatus for determining a torque angle for a synchronous electric motor of a mechanical circulatory support device are provided. The method includes receiving a voltage signal representing a voltage induced in a winding of the synchronous electric motor during operation of the synchronous electric motor, determining a first back electro-motive force (bEMF) value of the voltage signal within at least one time window during which the winding is not energized, determining a torque angle for the synchronous electric motor based, at least in part, on the first bEMF value, and outputting an indication of the torque angle.

### BACKGROUND

Cardiovascular diseases are a leading cause of morbidity, mortality, and burden on global healthcare. A variety of treatment modalities have been developed for heart health, ranging from pharmaceuticals to mechanical devices and transplantation. Temporary cardiac support devices, such as heart pump systems (also referred to as “intracardiac blood pumps”), provide hemodynamic support and facilitate heart recovery. Intracardiac blood pumps have traditionally been used to temporarily assist the pumping function of a patient’s heart during emergent cardiac procedures, such as a stent placement, performed after the patient suffers a heart attack, cardiac arrest, and/or cardiogenic shock. Intracardiac blood pumps also may be used to take the load off of a patient’s heart to allow the heart to recover from such a cardiac procedure or from a heart attack, cardiac arrest, cardiogenic shock, or heart damage (e.g., caused by a viral infection). In that regard, an intracardiac blood pump can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient’s body via an elongate drive shaft (or drive cable) or by an onboard motor located inside the patient’s body. Examples of such devices include the Impella^{®} family of devices (Abiomed, Inc., Danvers, MA).

### SUMMARY

Described herein are systems and methods for determining a torque angle of an electric motor (e.g., a permanent magnet synchronous motor (PMSM)), where the electric motor may be used to drive operation of a mechanical circulatory support (MCS) device (e.g., a heart pump). Some of the mechanical power generated by an electric motor is lost to heat, such that the actual torque provided by the motor is less than the torque that could be generated by an idealized motor. For motors that operate in a static (e.g., constant speed) condition, the amount of mechanical power lost to heat may be relatively constant as the motor rotates. However, for motors that operate in a pulsatile (e.g., changing speed) condition, such as a motor used to operate an MCS device, the load felt by the motor that the motor must overcome changes as the motor rotates. Accordingly, the amount of driving current that is converted to output mechanical power of the motor varies as the load changes. Additionally, the motor current measured at different times during rotation of a motor for a pulsatile driving scheme may be associated with different amounts of mechanical power lost to heat versus the mechanical power (torque) that is delivered by the motor. The inventor has recognized and appreciated that algorithms for an MCS device that use measurements of the motor current to estimate various quantities (e.g., pressures, device positioning) may benefit from more precise and/or additional information about the motor operation than conventional techniques provide.

Driving schemes for driving an electric motor for an MCS device may utilize six-step commutation in which two of three armature windings of the stator in a three-phase motor are sequentially energized (e.g., pulled to a positive voltage or ground), while the third winding has a floating potential. Pulse Amplitude Modulation (PAM) is a control technique in which the output voltage at a variable power supply (e.g., a three-phase supply) is modulated rather than requiring switching at the driver circuitry. In synchronous motors, the torque angle (also referred to herein as the “load angle”), which relates to the torque (mechanical power) provided by the motor, is the angle between the rotor magnetic field and the stator magnetic field induced by the current in the armature windings during motor operation. The rotor magnetic field induces a voltage in the unenergized winding as a result of a back electro-motive force or back EMF (bEMF), which can be measured and used to determine the rotor position relative to the stator. The torque angle of the motor can be estimated by observing the lag between the armature winding current and the voltage in the winding induced by bEMF. Driving circuitry for a synchronous motor may use the estimate of the torque angle to ensure that the motor is driven in a manner that keeps the torque angle within a range (e.g., slightly above 0 degrees) that maintains synchronous operation of the motor. Some conventional techniques for estimating the torque angle integrate voltage values induced in the unenergized winding as a result of bEMF. Such techniques output a number that represents the torque angle, but do not determine the torque angle itself. Some embodiments of the present disclosure relate to techniques for estimating the torque angle of the motor based, at least in part, on a window of voltage/current values induced in the unenergized coil due to bEMF.

In some embodiments, a mechanical circulatory support device is provided. The mechanical circulatory support device includes a rotor, a synchronous electric motor coupled to the rotor, a drive circuit configured to drive operation of the synchronous electric motor to rotate the rotor in a pulsatile condition, and at least one hardware processor. The at least one hardware processor is configured to receive a voltage signal representing a voltage induced in a winding of the synchronous electric motor during operation of the synchronous electric motor, determine a first back electro-motive force (bEMF) value of the voltage signal within at least one time window during which the winding is not energized, determine a torque angle for the synchronous electric motor based, at least in part, on the first bEMF value, and control an operation of the mechanical circulatory support device based, at least in part, on the torque angle.

In one aspect, the drive circuit is configured to drive operation of the synchronous electric motor according to a six step commutation driving scheme with each step of the six step commutation driving scheme corresponding to a 60 degree rotation of the rotor relative to a stator, and the at least one time window includes a first time window corresponding to a first step of the six step commutation driving scheme during which the winding is not energized. In another aspect, the first time window is centered on a first expected bEMF crossing point during the first step, and the first time window has a duration less than the duration of the first step. In another aspect, the duration of the first time window corresponds to less than a 30 degree rotation of the rotor relative to the stator. In another aspect, determining a first bEMF value of the voltage signal within at least one time window during which the winding is not energized comprises determining an average value of the voltage signal within the first time window. In another aspect, the at least one hardware processor is further configured to determine a bEMF amplitude value based on a bEMF constant for the synchronous electric motor and a speed of the synchronous electric motor, and determine a unit circle equivalent sinusoidal value by dividing the first bEMF value by the bEMF amplitude value, and determining the torque angle for the synchronous electric motor based, at least in part, on the first bEMF value comprises determining the torque angle as an arcsin of the unit circle equivalent sinusoidal value.

In another aspect, the at least one time window further includes a second time window corresponding to a second step of the six step commutation driving scheme during which the winding is not energized, and the at least one hardware processor is further configured to determine a second bEMF value of the voltage signal within the second time window, and determine the torque angle for the synchronous electric motor based, at least in part, on the first bEMF value and the second bEMF value. In another aspect, the second time window is centered on a second expected bEMF crossing point during the second step, and the second time window has a duration less than the duration of the second step. In another aspect, determining a second bEMF value of the voltage signal within at least one time window during which the winding is not energized comprises determining an average value of the voltage signal within the second time window. In another aspect, the six step commutation driving scheme comprises a pulse amplitude modulation driving scheme.

In another aspect, controlling an operation of the mechanical circulatory support device based, at least in part, on the torque angle comprises deriving one or more conditions associated with the mechanical circulatory support device based, at least in part, on the torque angle. In another aspect, deriving one or more conditions associated with the mechanical circulatory support device comprises determining a placement signal for the mechanical circulatory support device based, at least in part, on the torque angle, and controlling an operation of the mechanical circulatory support device based, at least in part, on the torque angle further comprises outputting on a user interface, an indication of the placement signal. In another aspect, determining a placement signal for the mechanical circulatory support device based, at least in part, on the torque angle comprises generating an adjusted motor current signal based on the torque angle, and determining the placement signal based, at least in part, on the adjusted motor current signal.

In some embodiments, a method of determining a torque angle for a synchronous electric motor is provided. The method includes receiving a voltage signal representing a voltage induced in a winding of the synchronous electric motor during operation of the synchronous electric motor, determining a first back electro-motive force (bEMF) value of the voltage signal within at least one time window during which the winding is not energized, determining a torque angle for the synchronous electric motor based, at least in part, on the first bEMF value, and outputting an indication of the torque angle.

In one aspect, the at least one time window includes a first time window corresponding to a first step of a six step commutation driving scheme during which the winding is not energized. In another aspect, determining a first bEMF value of the voltage signal within at least one time window during which the winding is not energized comprises determining an average value of the voltage signal within the first time window. In another aspect, the method further includes determining a bEMF amplitude value based on a bEMF constant for the synchronous electric motor and a speed of the synchronous electric motor, and determining a unit circle equivalent sinusoidal value by dividing the first bEMF value by the bEMF amplitude value, wherein determining the torque angle for the synchronous electric motor based, at least in part, on the first bEMF value comprises determining the torque angle as an arcsin of the unit circle equivalent sinusoidal value.

In another aspect, the at least one time window further includes a second time window corresponding to a second step of the six step commutation driving scheme during which the winding is not energized, and the method further includes determining a second bEMF value of the voltage signal within the second time window, and determining the torque angle for the synchronous electric motor based, at least in part, on the first bEMF value and the second bEMF value. In another aspect, determining a second bEMF value of the voltage signal within at least one time window during which the winding is not energized comprises determining an average value of the voltage signal within the second time window.

In another aspect, the synchronous electric motor is included in a mechanical circulatory support device, and the method further includes controlling an operation of the mechanical circulatory support device based, at least in part, on the torque angle. In another aspect, controlling an operation of the mechanical circulatory support device comprises deriving one or more conditions associated with the mechanical circulatory support device based, at least in part, on the torque angle. In another aspect, deriving one or more conditions associated with the mechanical circulatory support device comprises determining a placement signal for the mechanical circulatory support device based, at least in part, on the torque angle, and controlling an operation of the mechanical circulatory support device based, at least in part, on the torque angle further comprises outputting on a user interface, an indication of the placement signal. In another aspect, determining a placement signal for the mechanical circulatory support device based, at least in part, on the torque angle comprises generating an adjusted motor current signal based on the torque angle, and determining the placement signal based, at least in part, on the adjusted motor current signal.

### BRIEF DESCRIPTION OF DRAWINGS

FIG. 1 shows an illustrative heart pump device that may be used, in accordance with some embodiments.

FIGS. 2A-2C show example plots of a voltage waveform and a bEMF waveform associated with energizing a motor winding using a Pulse Amplitude Modulated (PAM) driving scheme where the motors are driven to have different torque angles, in accordance with some embodiments.

FIG. 3 is a representation of a technique for estimating a torque angle of a synchronous electric motor, in accordance with some embodiments.

FIG. 4 is a flowchart of a process for estimating a torque angle of a synchronous electric motor, in accordance with some embodiments.

### DETAILED DESCRIPTION

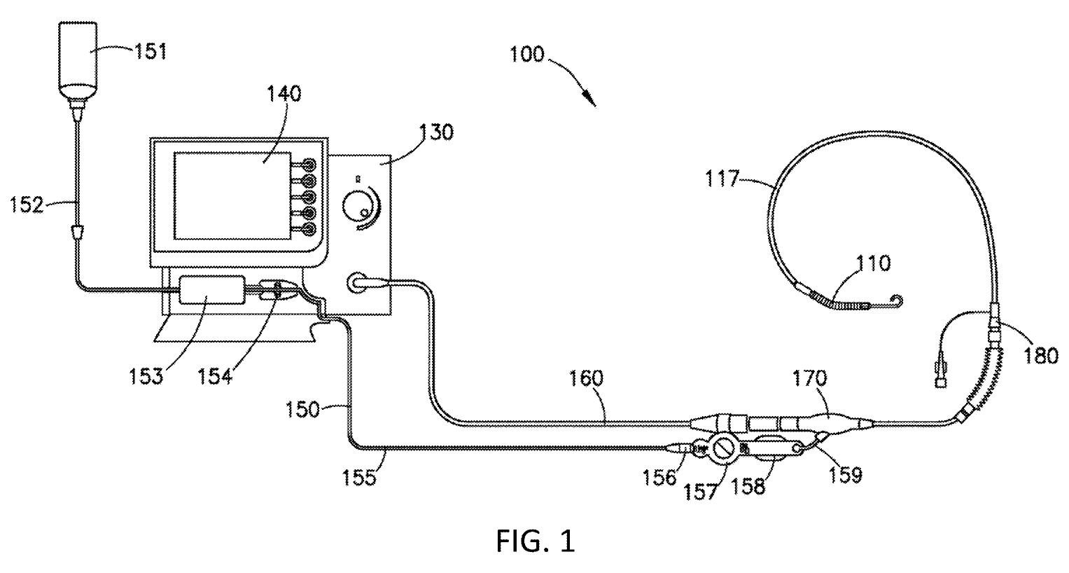

A circulatory support device (also referred to herein as a “heart pump” or simply a “pump”) may include a percutaneous, catheter-based device that provides hemodynamic support to the heart of a patient. As shown in FIG. 1, heart pump 110 may form part of a cardiac support system 100. Cardiac support system 100 also may include a controller 130 (e.g., an Automated Impella Controller^{®}, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of heart pump 110. During operation, purge subsystem 150 may be configured to deliver a purge fluid to heart pump 110 through catheter tube 117 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between heart pump 110 and controller 130. Plug 170 may connect catheter tube 117, purge subsystem 150, and connector cable 160. In some implementations, plug 170 may include a storage device (e.g., a memory) configured to store, for example, operating parameters to facilitate transfer of the patient to another controller if needed. Repositioning unit 180 may be used to reposition heart pump 110 in the patient’s heart.

As shown in FIG. 1, in some embodiments, the cardiac support system 100 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 100 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to heart pump 110. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 includes a flexible rubber diaphragm that provides the additional filling volume by means of an expansion chamber. Infusion filter 158 may help prevent bacterial contamination and air from entering catheter tube 117. Sidearm 159 may provide a fluidic connection between infusion filter 158 and plug 170. Although shown as having separate purge tubing and connector cable, it will be appreciated that in some embodiments, the cardiac support system 100 may include a single connector with both fluidic and electric lines connectable to the controller 130.

During operation, controller 130 may be configured to receive measurements from one or more pressure sensors (not shown) included as a portion of heart pump 110 and purge disc 154. Controller 130 may also be configured to control operation of the motor (not shown) of the heart pump 110 and purge cassette 153. For example, controller 130 may be configured to control drive circuitry for the motor to modify how the motor is operating (e.g., by changing the speed and/or torque output of the motor as desired). As noted herein, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter tube 117 and plug 170. Sensor cables (not shown) within catheter tube 117, connector cable 160, and plug 170 may provide an electrical connection between components of the heart pump 110 (e.g., one or more pressure sensors) and controller 130. Motor cables (not shown) within catheter tube 117, connector cable 160, and plug 170 may provide an electrical connection between the motor of the heart pump 110 and controller 130. During operation, controller 130 may be configured to receive measurements from one or more pressure sensors of the heart pump 110 through the sensor cables (e.g., optical fibers) and to control the electrical power delivered to the motor of the heart pump 110 through the motor cables. By controlling the power delivered to the motor of the heart pump 110, controller 130 may be operable to control the speed of the motor.

Various modifications can be made to cardiac support system 100 and one or more of its components. For instance, one or more additional sensors may be added to heart pump 110. In another example, a signal generator may be added to cardiac support system 100 to generate a signal indicative of the rotational speed of the motor of the heart pump 110. As another example, one or more components of cardiac support system 100 may be separated. For instance, display 140 may be incorporated into another device in communication with controller 130 (e.g., wirelessly or through one or more electrical cables).

As described herein, a heart pump 110 may include a motor configured to drive rotation of an impeller that causes blood to flow from an inlet of the heart pump 110 to an outlet of the heart pump 110. Such a pumping action enables blood to be transported across one or more heart valves when the heart pump 110 is properly positioned within a patient’s heart. The motor may be driven by a motor drive circuit (also referred to herein simply as a “drive circuit”).

The inventor has recognized that for a synchronous motor that includes permanent magnets, synchronization between the driving magnetic field and the internal magnetic field caused by the magnets in the motor spinning should be maintained to ensure proper and/or efficient operation of the motor. The current driven through the armature windings on the stator may result in a stator magnetic field. As the rotor rotates relative to the stator during operation, the spinning magnets on the rotor result in a changing magnetic field. A controller may include drive circuitry configured to maintain synchronicity of the motor by driving the motor in a manner that ensures the torque angle between the rotor magnetic field and the stator magnetic field remains within a desired range (e.g., between 0 and 90 degrees) during operation. As described above, in a six step commutation driving scheme, two of the three windings of a three phase motor may be sequentially energized while the third winding is floating. As the magnets in the rotor of the motor rotate, the rotation of the magnets induces a voltage in the motor windings, which may be referred to as a back electro-motive force (bEMF). The bEMF induced in the windings opposes the supply voltage and can be measured, the bEMF being representative of the rotor position relative to the stator.

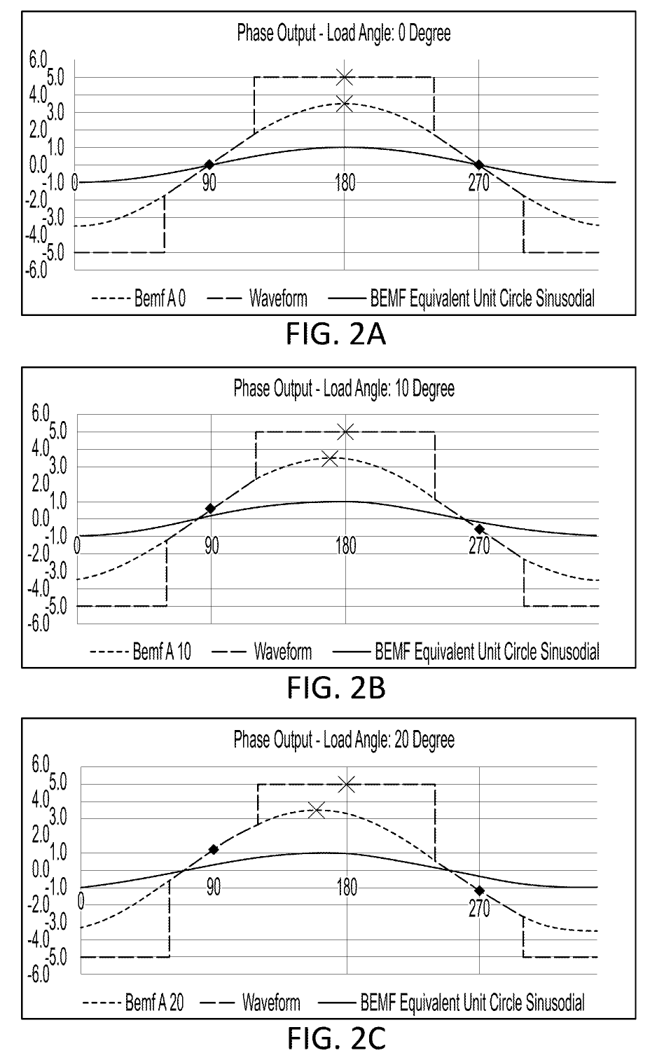

In an idealized motor operation, the voltage signal induced in the unenergized coil due to bEMF would align (i.e., have no lag) in phase with the current signal driving the windings (also referred to herein as the “armature current”). An example of a bEMF voltage signal induced in a winding according to such an idealized motor case is shown in FIG. 2A, in which the torque angle is 0 degrees meaning that the unenergized winding voltage waveform and armature current waveform are perfectly in phase. However, nonidealized motor operation may result in a lag (torque angle) between the phases of the winding voltage signal due to bEMF and the armature current waveform, examples of which are shown in FIGS. 2B-2C. In the example shown in FIG. 2B, the torque angle is 10 degrees, and in the example shown in FIG. 2C, the torque angle is 20 degrees.

As described above, some conventional techniques for estimating the torque angle based on a voltage/current signal induced by bEMF do not consider that, in a pulsatile control scheme used for operating a motor for an MCS device, the torque angle of the motor may continuously change as the motor rotates. For example, at some points in time during rotation the motor may experience more load, and at other points in time during the motor may experience less load, with the torque angle changing in unknown ways. Using such conventional techniques to estimate torque angle yields a number that represents the torque angle (e.g., due to integration of the unenergized winding signal due to bEMF) rather than being an estimation of the torque angle itself. Some embodiments described herein more fully characterize the operation of a synchronous motor compared with some conventional techniques by averaging values of the winding voltage within a window centered on an expected bEMF zero crossing point and using linear approximation at the expected point to determine the load angle.

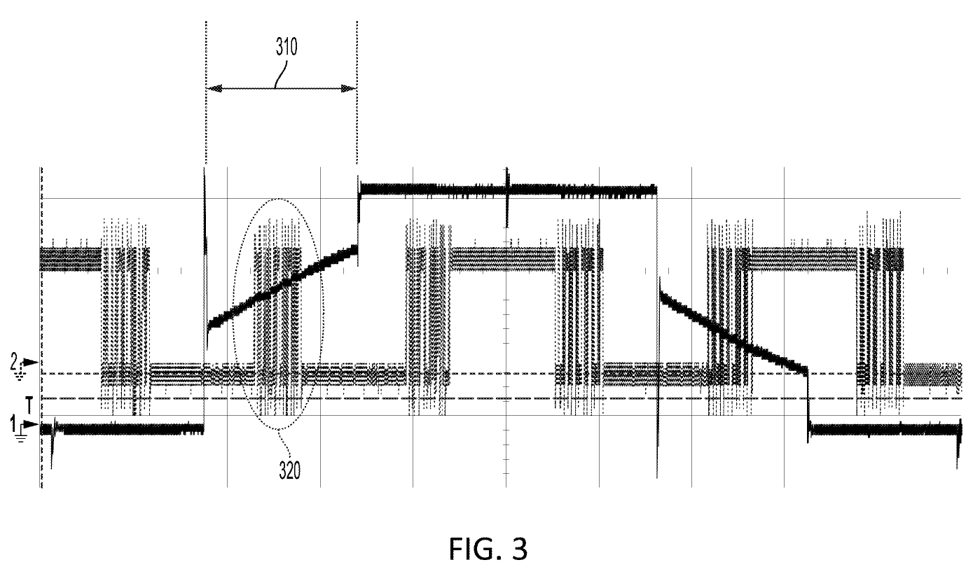

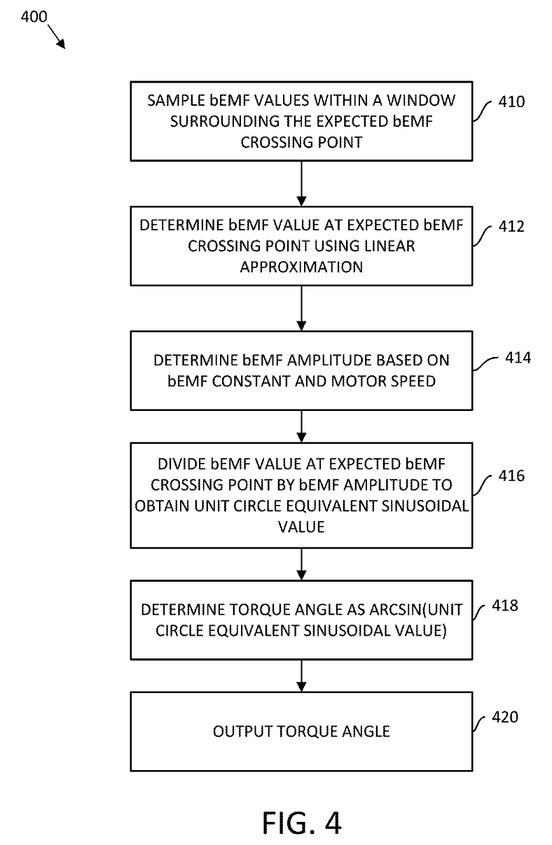

FIG. 3 schematically illustrates a voltage waveform induced in a winding of a three-phase motor controlled using a six step commutation driving scheme (e.g., PAM), in accordance with some embodiments. For example, if a full 360 degree rotation of the rotor about the stator is divided into six steps, each step corresponds to a 60 degree rotation of the rotor relative to the stator. As shown in FIG. 3, during a time period 310 during which the winding is not being excited, a voltage induced in the winding due to bEMF may be measured. In some embodiments, the values of the voltage signal within a time window 320 corresponding to a portion of time period 310 may be used to estimate the torque angle of the motor. The time window 320 may be centered on the expected bEMF crossing (e.g., if the system was operating at a 0 degree torque angle). Any suitable width of time window 320 may be used. As should be appreciated, during the transitions between states, the voltage waveform may exhibit transient noise spikes, and the time window 320 may be selected to be narrow enough to not include such noise information. For instance, time window 320 may correspond to 10 degrees of rotation (e.g., 1/6 of time period 310), 20 degrees of rotation (e.g., 1/3 of time period 310), 30 degrees of rotation (e.g., 1/2 of time period 310) or may have any other suitable width. As described below in connection with the method of FIG. 4, the winding voltage signal may be sampled within time window 320 and the sampled values may be used to determine the torque angle. As shown in FIG. 3, the voltage signal may be determined for two of the six steps of the commutation driving scheme (a high to low transition and a low to high transition) during which the winding is not energized (also referred to as the winding being in a tri-state).

FIG. 4 is a flowchart of a method 400 for determining a torque angle for a motor, in accordance with some embodiments. Method 400 may start in act 410, where voltage values associated with an unenergized motor winding are sampled within a window surrounding an expected bEMF crossing point as described above. Method 400 may then proceed to act 412, where the bEMF value at the expected bEMF crossing point may be determined using linear approximation. For example, the bEMF value at the expected crossing point may be determined as the average of the voltage values in the window of the sampled voltage signal. A linear approximation of the bEMF value at the expected crossing point may a valid approach since the expected operating region is likely to be in the roughly “linear” region of the sinusoidal bEMF wave as shown, for example, in FIGS. 2A-2C. Selection of a narrower analysis window may further ensure that the window falls within the linear region of the sinusoidal bEMF wave, at the expense of having fewer voltage values used to calculate the average. Using an average value of the voltage signal over a window rather than using a single value of the voltage signal at the expected crossing point may help reduce errors that may be present at any single point in time in the voltage signal. Method 400 may then proceed to act 414, where the bEMF amplitude is determined based on the bEMF constant for the motor and the motor speed. Method 400 may then proceed to act 416, where the bEMF value determined in act 412 is divided by the bEMF amplitude determined in act 414 to obtain a unit circle equivalent sinusoidal value (e.g., the value on a sinusoid that oscillates between peak values of -1 and 1). Method 400 may then proceed to act 418, where the torque angle for the motor is determined as the arcsin of the unit circle equivalent sinusoidal value determined in act 416. Method 400 may then proceed to act 420 where the torque angle is output. For example, the torque angle may be used to determine the actual mechanical power output by the motor (e.g., total mechanical power – mechanical power lost due to heat). As described herein, the torque angle may be used by one or more algorithms implemented by an MCS device that use motor current to derive one or more conditions associated with the MCS device, such as an indirect pressure measurement or an MCS device placement signal. It should be appreciated however, that a torque angle determined in accordance with the techniques described herein may be used for any other suitable purpose.

Having thus described several aspects and embodiments of the technology set forth in the disclosure, it is to be appreciated that various alterations, modification, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1. A mechanical circulatory support device, comprising:
a rotor;
a synchronous electric motor coupled to the rotor;
a drive circuit configured to drive operation of the synchronous electric motor to rotate the rotor in a pulsatile condition; and
at least one hardware processor configured to:
receive a voltage signal representing a voltage induced in a winding of the synchronous electric motor during operation of the synchronous electric motor;
determine a first back electro-motive force (bEMF) value of the voltage signal within at least one time window during which the winding is not energized;
determine a torque angle for the synchronous electric motor based, at least in part, on the first bEMF value; and
control an operation of the mechanical circulatory support device based, at least in part, on the torque angle.

2. The mechanical circulatory support device of claim 1, wherein
the drive circuit is configured to drive operation of the synchronous electric motor according to a six step commutation driving scheme with each step of the six step commutation driving scheme corresponding to a 60 degree rotation of the rotor relative to a stator, and
the at least one time window includes a first time window corresponding to a first step of the six step commutation driving scheme during which the winding is not energized.

3. The mechanical circulatory support device of claim 2, wherein
the first time window is centered on a first expected bEMF crossing point during the first step, and
the first time window has a duration less than the duration of the first step.

4. The mechanical circulatory support device of claim 3, wherein the duration of the first time window corresponds to less than a 30 degree rotation of the rotor relative to the stator.

5. The mechanical circulatory support device of claim 2, wherein determining a first bEMF value of the voltage signal within at least one time window during which the winding is not energized comprises determining an average value of the voltage signal within the first time window.

6. The mechanical circulatory support device of claim 5, wherein the at least one hardware processor is further configured to:
determine a bEMF amplitude value based on a bEMF constant for the synchronous electric motor and a speed of the synchronous electric motor; and
determine a unit circle equivalent sinusoidal value by dividing the first bEMF value by the bEMF amplitude value,
wherein determining the torque angle for the synchronous electric motor based, at least in part, on the first bEMF value comprises determining the torque angle as an arcsin of the unit circle equivalent sinusoidal value.

7. The mechanical circulatory support device of claim 2, wherein
the at least one time window further includes a second time window corresponding to a second step of the six step commutation driving scheme during which the winding is not energized, and
the at least one hardware processor is further configured to:
determine a second bEMF value of the voltage signal within the second time window; and
determine the torque angle for the synchronous electric motor based, at least in part, on the first bEMF value and the second bEMF value.

8. The mechanical circulatory support device of claim 7, wherein
the second time window is centered on a second expected bEMF crossing point during the second step, and
the second time window has a duration less than the duration of the second step.

9. The mechanical circulatory support device of claim 8, wherein determining a second bEMF value of the voltage signal within at least one time window during which the winding is not energized comprises determining an average value of the voltage signal within the second time window.

10. The mechanical circulatory support device of claim 1, wherein controlling an operation of the mechanical circulatory support device based, at least in part, on the torque angle comprises deriving one or more conditions associated with the mechanical circulatory support device based, at least in part, on the torque angle.

11. The mechanical circulatory support device of claim 10, wherein
deriving one or more conditions associated with the mechanical circulatory support device comprises determining a placement signal for the mechanical circulatory support device based, at least in part, on the torque angle, and
controlling an operation of the mechanical circulatory support device based, at least in part, on the torque angle further comprises outputting on a user interface, an indication of the placement signal.

12. The mechanical circulatory support device of claim 11, wherein determining a placement signal for the mechanical circulatory support device based, at least in part, on the torque angle comprises generating an adjusted motor current signal based on the torque angle, and determining the placement signal based, at least in part, on the adjusted motor current signal.

13. A method of determining a torque angle for a synchronous electric motor, the method comprising:
receiving a voltage signal representing a voltage induced in a winding of the synchronous electric motor during operation of the synchronous electric motor;
determining a first back electro-motive force (bEMF) value of the voltage signal within at least one time window during which the winding is not energized;
determining a torque angle for the synchronous electric motor based, at least in part, on the first bEMF value; and
outputting an indication of the torque angle.

14. The method of claim 13, wherein the at least one time window includes a first time window corresponding to a first step of a six step commutation driving scheme during which the winding is not energized.

15. The method of claim 14, wherein determining a first bEMF value of the voltage signal within at least one time window during which the winding is not energized comprises determining an average value of the voltage signal within the first time window.

16. The method of claim 15, further comprising:
determining a bEMF amplitude value based on a bEMF constant for the synchronous electric motor and a speed of the synchronous electric motor; and
determining a unit circle equivalent sinusoidal value by dividing the first bEMF value by the bEMF amplitude value,
wherein determining the torque angle for the synchronous electric motor based, at least in part, on the first bEMF value comprises determining the torque angle as an arcsin of the unit circle equivalent sinusoidal value.

17. The method of claim 14, wherein
the at least one time window further includes a second time window corresponding to a second step of the six step commutation driving scheme during which the winding is not energized, and
the method further comprises:
determining a second bEMF value of the voltage signal within the second time window; and
determining the torque angle for the synchronous electric motor based, at least in part, on the first bEMF value and the second bEMF value.

18. The method of claim 17, wherein determining a second bEMF value of the voltage signal within at least one time window during which the winding is not energized comprises determining an average value of the voltage signal within the second time window.

19. The method of claim 13, wherein the synchronous electric motor is included in a mechanical circulatory support device, the method further comprising:
controlling an operation of the mechanical circulatory support device based, at least in part, on the torque angle, wherein controlling an operation of the mechanical circulatory support device comprises deriving one or more conditions associated with the mechanical circulatory support device based, at least in part, on the torque angle.

20. The method of claim 19, wherein
deriving one or more conditions associated with the mechanical circulatory support device comprises determining a placement signal for the mechanical circulatory support device based, at least in part, on the torque angle, and
controlling an operation of the mechanical circulatory support device based, at least in part, on the torque angle further comprises outputting on a user interface, an indication of the placement signal,
wherein determining a placement signal for the mechanical circulatory support device based, at least in part, on the torque angle comprises generating an adjusted motor current signal based on the torque angle, and determining the placement signal based, at least in part, on the adjusted motor current signal.
